(19) United States
(12) Patent Application Publication
(10) Pub. No.: US 2025/0000333 A1
(43) Pub. Date: Jan. 2, 2025

(54) ENDOSCOPE SENSOR INTERFERENCE MITIGATION

(71) Applicant: Lumenis, Ltd., Yokneam Ilit (IL)

(72) Inventor: Tal Waisman, Haifa (IL)

(73) Assignee: Lumenis, Ltd., Yokneam llit (IL)

(21) Appl. No.: 18/758,625

(22) Filed: Jun. 28, 2024

Related U.S. Application Data
(60) Provisional application No. 63/511,430, filed on Jun. 30, 2023.

(51) Int. Cl. A61B 1/00; A61B 18/26

(52) U.S. Cl. CPC A61B 1/00097; A61B 1/00006; A61B 18/26

(57) ABSTRACT

Described herein is an endoscopic system for use with a laser source. The endoscopic system is configured to synchronize measurement, captured by an endoscopic sensor, of physical characteristics of an environment with lasing by the laser source. The endoscopic system is further configured to modify measurements captured by the endoscopic sensor based on lasing by the laser source.

### CROSS-REFERENCE TO RELATED APPLICATIONS

This application claims the benefit of U.S. Provisional Patent Application Ser. No. 63/511,430 filed on Jun. 30, 2023, the disclosure of which is incorporated herein by reference.

### TECHNICAL FIELD

The present disclosure generally relates to endoscopes and particularly, but not exclusively, to mitigating interference with endoscope sensors when used in conjunction with a surgical laser system.

### BACKGROUND

Endoscopes are used to obtain an internal view of a patient. Further, endoscopes often include multiple working channels in which various surgical devices and tools can be inserted. For example, during a lithotripsy procedure, a fiber optic cable is inserted through a working channel of an endoscope (or ureteroscope to be precise) and light from the fiber optic cable can be used to ablate a target (e.g., stone, or the like). Endoscopes are often equipped with sensors or include additional working channels through which sensors can be positioned in the treatment environment to monitor conditions of the environment.

Conventionally, such sensors cannot be used during active lasing. However, there is a need to mitigate interference with sensor measurements and provide for the use of sensors during a procedure and/or in conjunction with lasing.

### BRIEF SUMMARY

This summary is provided to introduce a selection of concepts in a simplified form that are further described below in the Detailed Description. This summary is not intended to necessarily identify key features or essential features of the claimed subject matter, nor is it intended as an aid in determining the scope of the claimed subject matter.

The present disclosure provides an endoscopic laser lithotripsy device and methods for such a device where sensor use and/or sensor measurements can be acquired without interference from the laser.

The embodiments described herein can be implemented as a method for an endoscope system. The method can comprise receiving, at a processor of an endoscope console, an indication of a pulse start control signal and one or more laser pulse characteristics, the pulse start control signal to define an initiation of lasing by the lasing console and the one or more laser pulse characteristics to define the lasing; determining, by the processor, a measurement time period for a sensor of the endoscope system based on the pulse start control signal and the one or more laser pulse characteristics; and receiving by the processor or generating by the processor, sensor measurements based on the measurement time period.

In further embodiments of the method, receiving by the processor sensor measurements based on the measurement time period comprising generating a control signal, the control signal to cause the sensor to measure a physical characteristic of an environment in which the endoscope is disposed based on the measurement time period; and receiving, at the processor from the sensor, an information element comprising indications of the physical characteristic measured based on the measurement time period.

In further embodiments of the method, the control signal comprises an indication of the measurement time period and an indication to measure the physical characteristic outside the measurement time period.

In further embodiments of the method, the control signal comprises an indication to power gate the sensor during the measurement time period.

In further embodiments of the method, generating by the processor sensor measurements based on the measurement time period can comprise receiving, at the processor from the sensor, an information element comprising indications of physical characteristic of an environment measured by the sensor; and generating, by the processor, the sensor measurements based on the physical characteristics and the measurement time period.

In further embodiments of the method, generating, by the processor, the sensor measurements based on the physical characteristics and the measurement time period can comprise removing measurements captured during the measurement time period.

In further embodiments of the method, generating, by the processor, the sensor measurements based on the physical characteristics and the measurement time period can comprise normalizing measurements captured during the measurement time period based on measurements captured outside the measurement time period.

In further embodiments of the method, the one or more laser pulse characteristics define a frequency of pulses to be generated by the lasing console.

In further embodiments of the method, the one or more laser pulse characteristics define a pulse width of the pulses.

In further embodiments of the method, determining the measurement time period can comprise determining an initiation of one or more laser pulses based on the pulse start control signal; determining a pulse width of the one or more laser pulses based on the one or more laser pulse characteristics; determining a conclusion of the one or more laser pulses based on the pulse width; and determining the measurement time period based on the initiation and conclusion of the one or more laser pulses.

In further embodiments of the method, determining the measurement time period can comprise determining a first instance where an intensity of one or more laser pulses exceeds a threshold value; determining a second instance subsequent to the first instance where an intensity of the one or more laser pulses does not exceed the threshold value; and determining the measurement time period based on the first instance and the second instance.

Embodiments of the disclosure can also be implemented as a computer readable storage device comprising instructions that when executed by a processor of an endoscopic system cause the endoscopic system to implement any one of the methods described herein.

Embodiments of the disclosure can be implemented as an endoscopic system comprising an endoscope comprising one or more working channels; an endoscopic sensor; and an endoscopic controller. The endoscopic controller can comprise a processor and a memory comprising instructions, which when executed by the processor cause the processor to implement any of the methods described herein.

In further embodiments of the endoscopic system, the lasing console comprises a Thulium fiber laser source or a Holmium laser source.

In further embodiments of the endoscopic system, the sensor is to be inserted into a working channel of the endoscope.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A, FIG. 1B, FIG. 1C, and FIG. 1D illustrate a lithotripsy system in accordance with at least one embodiment of the present disclosure.

FIG. 2 illustrates an endoscope system in accordance with at least one embodiment of the present disclosure.

FIG. 3 illustrates a method of for a lithotripsy system in accordance with at least one embodiment of the present disclosure.

FIG. 4 illustrates another endoscope system in accordance with at least one embodiment of the present disclosure.

FIG. 5 illustrates another method of for a lithotripsy system in accordance with at least one embodiment of the present disclosure.

FIG. 6 and FIG. 7 illustrate plots showing aspects of the subject matter in accordance with at least one embodiment of the present disclosure.

FIG. 8 illustrates a computer-readable storage medium in accordance with at least one embodiment of the present disclosure.

FIG. 9 illustrates a diagrammatic representation of a machine in the form of a computer system within which a set of instructions may be executed for causing the machine to perform any one or more of the methodologies discussed herein, according to at least one embodiment of the present disclosure.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

As noted, the present disclosure provides systems and techniques to mitigate interference of endoscopic sensors used in conjunction with a laser. It will be appreciated that such interference can occur at various points during the timeline of treatment and can disrupt some sensor data while leaving other sensor data uninfluenced. Further, some sensors may be operable even when exposed to laser light while other sensors may not be operable (e.g., may saturate, or the like) when exposed to laser light.

As an example, medical lasers are used in a variety of endoscopic procedures where laser light is directed to a target through an optical fiber. One such procedure, to address renal calculi (e.g., kidney stones) is ureteral endoscopy, or lithotripsy. In lithotripsy, an endoscopic probe, with a camera and other sensors, is inserted into the patient's urinary tract to locate the calculi for removal. An optical fiber is inserted through a working channel of the probe and laser energy can be directed towards the calculi via the optical fiber to disintegrate the calculi as they are found via the camera.

During such a procedure, a physician will often monitor the intra-renal pressure caused by irrigation (e.g., via a pressure sensor either incorporated into the distal end of the endoscope or inserted through a working channel of the endoscope). Further, a physician will often monitor the intra-renal temperature of fluids in the environment in which the lithotripsy procedure takes place (e.g., via a pressure sensor either incorporated into the distal end of the endoscope or inserted through a working channel of the endoscope).

In general, the need is to take short-term average measurements of these physical conditions (e.g., pressure, temperature, etc.) However, many endoscopic procedures involve firing a laser in the vicinity of the sensors. It is to be appreciated that laser pulses will vaporize matter in the environment (e.g., water, stone, tissue, etc.) which can create very short, and possibly very large, alterations in the measured values. As an example, a laser pulse will vaporize liquid creating a vapor bubble, which will inflate as the pulse is fired and collapse once the pulse stops. The inflating and collapsing of the vapor bubble can cause pressure waves on the order of 10 bars. Further, temperatures at the boiling point of the bubble formation can be around 100° C. These extreme values can bias the average measurements. Further, these extreme values can saturate some sensors and/or the electronics, which could prevent correct readings for some additional period after the extreme values drop back to normal levels.

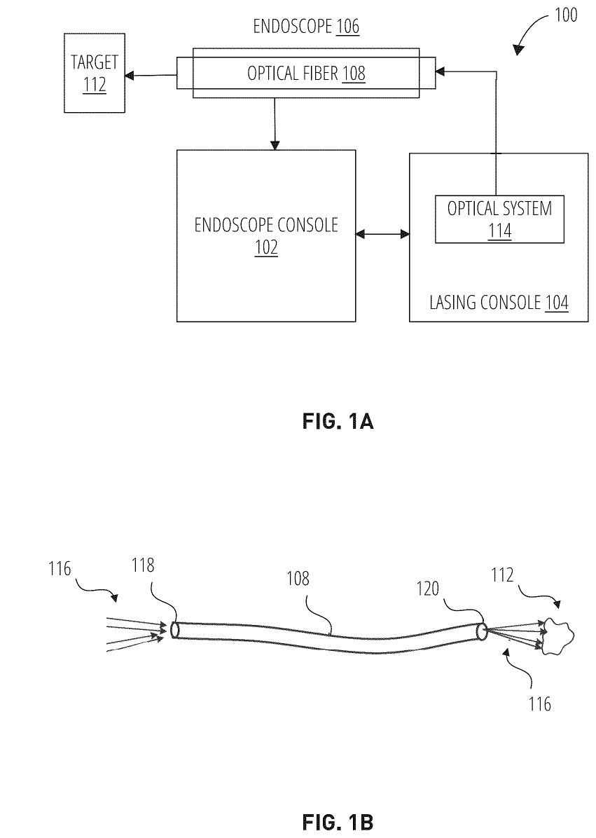

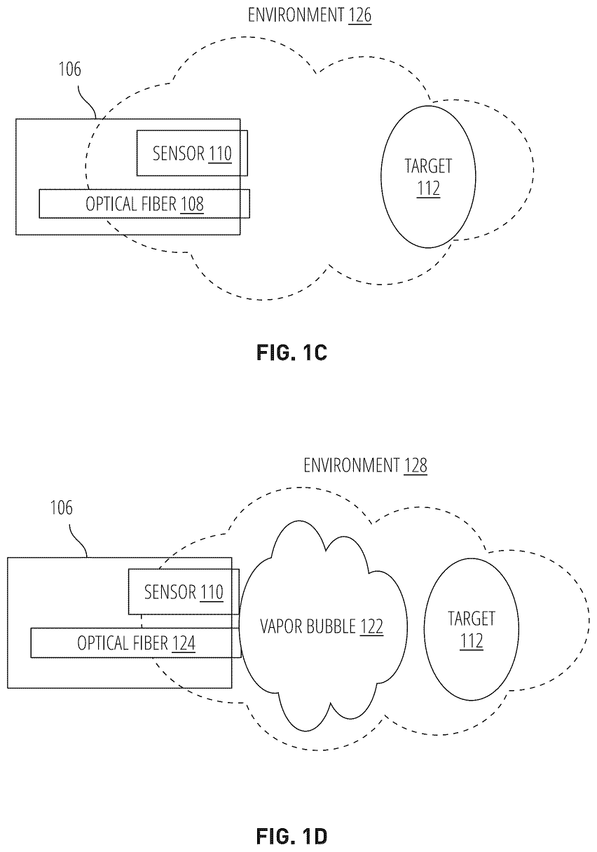

FIG. 1A to FIG. 1D show an exemplary lithotripsy system 100 configured to mitigate interference in sensor measurement. Lithotripsy system 100 can comprise an endoscope console 102 and a lasing console 104, coupled to an endoscope 106 and an optical fiber 108, respectively. The endoscope 106 can be any of a variety of “scopes” such as, for example, a ureteroscope, a colonoscope, a bronchoscope, or the like. The endoscope 106 can include one or more working channels in which the optical fiber 108 can be inserted. Further, endoscope 106 can include a sensor 110 and/or can include a working channel in which sensor 110 can be inserted (see FIG. 1C and FIG. 1D). Sensor 110 can be utilized to measure physical characteristics of the environment 126 in which the endoscope 106 is disposed. Further, these figures depict a target 112, upon which a procedure may be applied. In some embodiments, the target 112 may be a tissue, a stone, a tumor, a cyst, and the like, within a subject, which is to be treated, ablated, or destroyed. In some embodiments, the subject may be a human being or an animal.

Lasing console 104 can comprise an optical system 114 arranged to generate incident light 116 which can be directed to target 112 via optical fiber 108. As depicted more fully in FIG. 1B, the optical fiber 108 comprises a proximal end 118 and a distal end 120. The proximal end 118 is the end of the optical fiber 108 through which light beams enter while the distal end 120 is the end of the optical fiber 108 through which light beams are emitted and via which light beams can be directed onto the target 112. For example, this figure depicts incident light 116 entering the optical fiber 108 at the proximal end 118, propagating through length of the optical fiber 108, exiting the optical fiber 108 at the distal end 120, and being incident on the target 112 from the distal end 120 of the optical fiber 108.

Although not depicted here, optical system 114 can comprise one or more laser light sources and various optics arranged to generate incident light 116 and couple incident light 116 to optical fiber 108. As an example, the laser light sources may include, but are not limited to, solid-state lasers, gas lasers, diode lasers, and fiber lasers. The light beams may include one or more of an aiming beam, a treatment beam, and any other beam transmitted through the optical fiber 108. In various embodiments, an aiming beam may include a light beam of low intensity that is transmitted through the optical fiber 108 to illuminate or highlight the target 112 while a treatment beam may include a light beam of high intensity that is transmitted through the optical fiber 108 to treat the target 112. The various optics within optical system 114 can include, but are not limited to, one or more polarizers, beam splitters, beam combiners, light detector, wavelength division multiplexers, collimators, circulators, and/or lenses. The laser light sources of optical system 114 can comprise a Thulium fiber laser, a Holmium laser, or other types of laser light sources.

Although endoscope console 102 and lasing console 104 are depicted as separate consoles in FIG. 1A, it is to be appreciated that an embodiment could be implemented where endoscope console 102 and lasing console 104 are combined into a single console, for example, sharing many computing components (e.g., processor, memory, display, controls, etc.). However, for purposes of clarity, the disclosure describes examples where endoscope console 102 and lasing console 104 are separate.

Turning more particularly to FIG. 1C and FIG. 1D, sensor 110 of endoscope 106 is depicted. It is to be appreciated that although these figures and this description depict and reference a single sensor, endoscope 106 could be provided with multiple sensors and/or working channels in which one or more sensors can be inserted, or some combination of sensors embodied in the endoscope and working channels in which sensors can be inserted. The sensor 110 (or sensors 110 as may be the case) can be any of a variety of sensors, such as, a pressure sensor, a temperature sensor, or the like. As noted, sensor 110 can measure physical characteristics (e.g., temperature, pressure, or the like) of environment 126 during a procedure.

However, when a laser pulse of incident light 116 is emitted from optical fiber 108, vapor bubble 122 can be created, which can significantly change the physical characteristic of the environment 126, thereby biasing the measurement captured by sensor 110 or saturating the sensor 110 and affecting the ability to properly measure the physical characteristic.

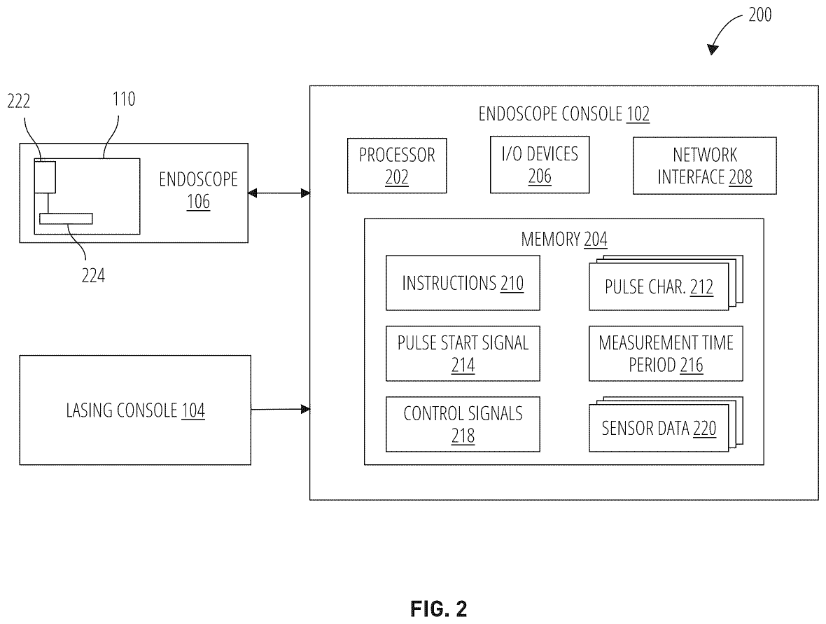

FIG. 2 illustrates an endoscope system 200, in accordance with non-limiting examples of the present disclosure. In general, endoscope system 200 is a system for measuring physical characteristics in an environment in which an endoscope is deployed while reducing measurement interference from lasing. Endoscope system 200 includes endoscope console 102 arranged to be coupled to lasing console 104 and endoscope 106. In general, during operation, endoscope console 102 is arranged to receive information about laser energy (e.g., laser beams, pulses of laser light, or the like) generated or to be generated by lasing console 104 and to synchronize measuring the physical characteristics by sensor 110 in endoscope 106 using the received information about the laser energy to reduce interference with the measurements and/or reduce or mitigate saturating the sensors with extreme values.

Endoscope console 102 can be any of a variety of computing devices. With some embodiments, endoscope console 102 can be a workstation, server, laptop, or tablet communicatively coupled to endoscope 106 and lasing console 104. Endoscope console 102 can include processor 202, memory 204, input and/or output (I/O) devices 206, and network interface 208. The processor 202 may include circuitry or processor logic, such as, for example, any of a variety of commercial processors. In some examples, processor 202 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 202 may include graphics processing portions and may include dedicated memory, multiple-threaded processing and/or some other parallel processing capability. In some examples, the processor 202 may be an application specific integrated circuit (ASIC) or a field programmable integrated circuit (FPGA).

The memory 204 may include logic, a portion of which includes arrays of integrated circuits, forming non-volatile memory to persistently store data or a combination of non-volatile memory and volatile memory. It is to be appreciated, that the memory 204 may be based on any of a variety of technologies. In particular, the arrays of integrated circuits included in memory 120 may be arranged to form one or more types of memory, such as, for example, dynamic random access memory (DRAM), NAND memory, NOR memory, or the like.

I/O devices 206 can be any of a variety of devices to receive input and/or provide output. For example, I/O devices 206 can include, a keyboard, a mouse, a joystick, a foot pedal, a display, a touch enabled display, a haptic feedback device, an LED, or the like.

Network interface 208 can include logic and/or features to support a communication interface. For example, network interface 208 may include one or more interfaces that operate according to various communication protocols or standards to communicate over direct or network communication links. Direct communications may occur via use of communication protocols or standards described in one or more industry standards (including progenies and variants). For example, network interface 208 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 208 can include logic and/or features to enable communication over a variety of wired or wireless network standards (e.g., 802.11 communication standards). For example, network interface 208 may be arranged to support wired communication protocols or standards, such as, Ethernet, or the like. As another example, network interface 208 may be arranged to support wireless communication protocols or standards, such as, for example, Wi-Fi, Bluetooth, ZigBee, LTE, 5G, or the like.

Memory 204 can include instructions 210, laser pulse characteristics 212, laser pulse start signal 214, measurement time period 216, control signals 218, and sensor data 220. In general, during operation, processor 202 can execute instructions 210 to cause endoscope console 102 to receive laser pulse characteristics 212 and laser pulse start signal 214 from lasing console 104. Laser pulse characteristics 212 can comprise an indication of the pulse repetition rate (e.g., laser pulse frequency, or the like) as well as the duration of the pulse (e.g., pulse width, or the like).

Additionally, processor 202 can execute instructions 210 to determine measurement time period 216 from laser pulse characteristics 212 and laser pulse start signal 214 and generate control signals 218 comprising an indication of measurement time period 216. Control signals 218 can comprise signals used to control the timing or time period in which sensor 110 measures the physical characteristics of the environment 126. Sensor 110 can include sensor circuitry and mechanicals 222 and sensor control circuitry 224. During operation, the sensor control circuitry 224 can control the sensor circuitry and mechanicals 222 to turn the sensor on and off and/or to control the capturing and readout of signals indicative of the physical characteristic such that the measurement aligns with measurement time period 216.

Accordingly, the present disclosure provides to generate control signals 218 comprising an indication for sensor control circuitry 224 to control when to turn on and off sensor circuitry and mechanicals 222 to synchronize the measurement of the physical characteristics of environment 126 with lasing by the lasing console 104. Said differently, sensor control circuitry 224 can use control signals 218 to cause sensor circuitry and mechanicals 222 to measure physical characteristics of environment 126 in a time period (e.g., measurement time period 216) when lasing console 104 is not emitting laser pulses from optical fiber 108 and/or when vapor bubble 122 is not expanding or collapsing based on laser pulse characteristics 212 and laser pulse start signal 214.

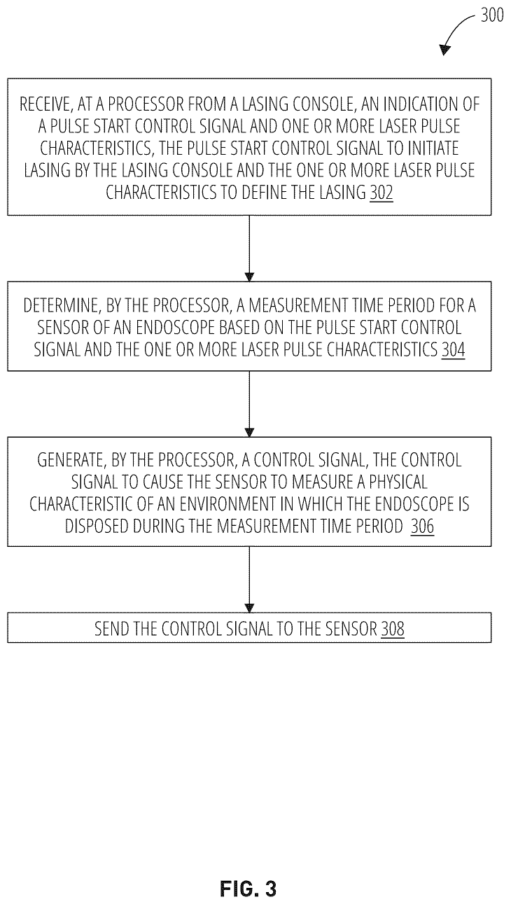

FIG. 3 illustrates a method 300 to synchronize the measurement of physical characteristics with a lasing console. For example, endoscope console 102 can implement method 300 to synchronize the measurement by sensor 110 with lasing by the lasing console 104. That is, processor 202 can execute instructions 210 to cause endoscope console 102 to implement method 300.

Method 300 can begin at block 302. At block 302 “receive, from a lasing console, an indication of a pulse start control signal and one or more laser pulse characteristics, the pulse start control signal to initiate lasing by the lasing console and the one or more laser pulse characteristics to define the lasing” a pulse start control signal and one or more laser pulse characteristics can be received from a lasing console. The pulse start control signal can define an initiation of lasing by the lasing console while the one or more laser pulse characteristics can define the lasing. For example, processor 202 can execute instructions 210 to receive laser pulse start signal 214 and laser pulse characteristics 212. As detailed above laser pulse start signal 214 can correspond to a signal that defines the initiation of lasing by the lasing console 104 while laser pulse characteristics 212 can define the lasing itself (e.g., pulse width, frequency, etc.)

Continuing to block 304 “determine, by the processor, a measurement time period for a sensor of an endoscope based on the pulse start control signal and the one or more laser pulse characteristics” a measurement time period for a sensor can be determined based on the pulse start control signal and the one or more laser pulse characteristics. For example, processor 202 can execute instructions 210 to determine measurement time period 216 based on laser pulse start signal 214 and laser pulse characteristics 212 where measurement time period 216 defines a period over which measurement by sensor 110 is to take place. As a specific example, processor 202 can execute instructions 210 to determine a period in which to activate sensor circuitry and mechanicals 222 when the lasing console 104 is not lasing.

Continuing to block 306 “generate, by the processor, a control signal, the control signal to cause the sensor to measure a physical characteristic of an environment in which the endoscope is disposed during the measurement time period” a control signal can be generated where the control signal is configured to cause a sensor to measure a physical characteristic of an environment during a period specified by the control signal. For example, processor 202 can execute instructions 210 to generate control signals 218 where control signals 218 can cause sensor circuitry and mechanicals 222 to actively measure a physical characteristic during the measurement time period 216. It is important to note that with some embodiments, control signals 218 can cause sensor control circuitry 224 to turn off and/or power gate sensor circuitry and mechanicals 222 such that sensor circuitry and mechanicals 222 will not be saturated when lasing console 104 lases.

Continuing to block 308 “send the control signal to the sensor” the control signal can be sent to the sensor. For example, processor 202 can execute instructions 210 to send the control signals 218 to the sensor 110 or rather, to sensor control circuitry 224 which can cause sensor circuitry and mechanicals 222 to activate based on measurement time period 216. The method 300 can further include blocks (not shown) where information elements comprising indications of measurement data can be received from the sensor, processed, and/or displayed on a display. For example, processor 202 can execute instructions 210 to receive sensor data 220 from sensor 110 (e.g., via a memory readout process, or the like) and can process the sensor data 220 to generate graphical indications of the measurements to display on a display coupled to the endoscope system 200. In particular, the method 300 can be implemented in real time, or rather, during a procedure, such that measurement of physical characteristics by sensor 110 of endoscope 106 can be synchronized with lasing by the lasing console 104 to reduce and/or mitigate interference with sensor operation.

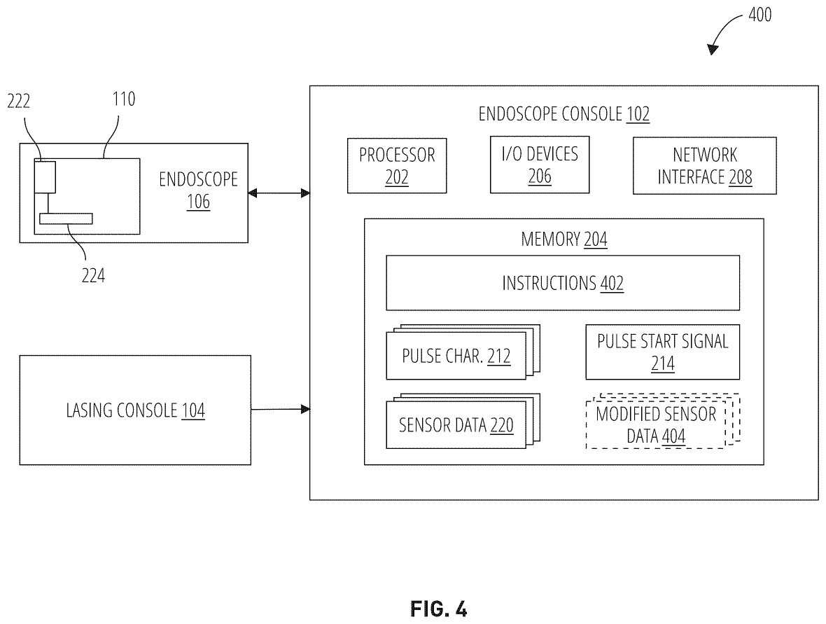

FIG. 4 illustrates an endoscope system 400, in accordance with non-limiting examples of the present disclosure. In general, endoscope system 400 is a system for measuring physical characteristics in an environment in which an endoscope is deployed while reducing measurement interference from lasing. Endoscope system 400 includes many of the components of endoscope system 200, including endoscope console 102 arranged to be coupled to lasing console 104 and endoscope 106. The difference being that endoscope system 400 is configured to receive sensor data from sensor 110 and generate modified sensor data or normalized sensor data based on adjusting the sensor data to account for interference from lasing by the lasing console 104.

To that end, memory 204 of endoscope system 400 can include instructions 402, laser pulse characteristics 212, laser pulse start signal 214, sensor data 220, and modified sensor data 404. In general, during operation, processor 202 can execute instructions 402 to cause endoscope console 102 to receive laser pulse characteristics 212 and laser pulse start signal 214 from lasing console 104 and to receive sensor data 220 from sensor 110. Like detailed above, laser pulse characteristics 212 can comprise an indication of the pulse repetition rate (e.g., laser pulse frequency, or the like) as well as the duration of the pulse (e.g., pulse width, or the like) and laser pulse start signal 214 can comprise an indication of a start to the lasing or a start of a laser pulse. Further, sensor data 220 can include indications of measurements of a physical characteristic or physical characteristics of environment 126.

Processor 202 can execute instructions 402 to generate modified sensor data 404 from sensor data 220 by removing portions of sensor data 220 based on laser pulse characteristics 212 and laser pulse start signal 214 and/or by normalizing sensor data 220 during periods indicated by laser pulse characteristics 212 and laser pulse start signal 214.

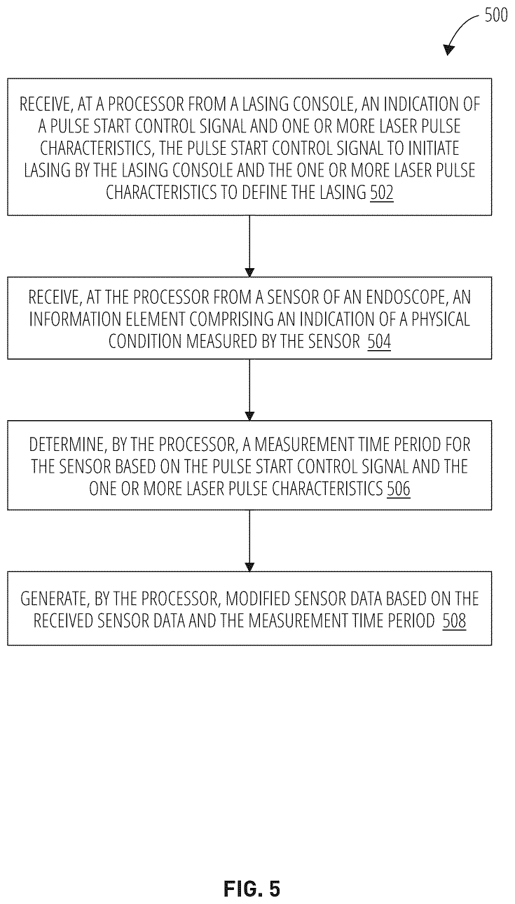

FIG. 5 illustrates a method 500 to synchronize the measurement of physical characteristics with a lasing console. For example, endoscope console 102 can implement method 500 to synchronize the measurements captured by sensor 110 with lasing by the lasing console 104. That is, processor 202 can execute instructions 210 to cause endoscope console 102 to implement method 500.

Method 500 can begin at block 502. At block 502 “receive, from a lasing console, an indication of a pulse start control signal and one or more laser pulse characteristics, the pulse start control signal to initiate lasing by the lasing console and the one or more laser pulse characteristics to define the lasing” a pulse start control signal and one or more laser pulse characteristics can be received from a lasing console. The pulse start control signal can define an initiation of lasing by the lasing console while the one or more laser pulse characteristics can define the lasing. For example, processor 202 can execute instructions 402 to receive laser pulse start signal 214 and laser pulse characteristics 212. As detailed above laser pulse start signal 214 can correspond to a signal that defines the initiation of lasing by the lasing console 104 while laser pulse characteristics 212 can define the lasing itself (e.g., pulse width, frequency, etc.)

Continuing to block 504 “receive, at the processor from a sensor of an endoscope, an information element comprising an indication of a physical condition measured by the sensor” information elements comprising indications of measurement data can be received from the sensor. For example, processor 202 can execute instructions 402 to receive sensor data 220 from sensor 110 (e.g., via a memory readout process, or the like).

Continuing to block 506 “determine, by the processor, a measurement time period for a sensor of an endoscope based on the pulse start control signal and the one or more laser pulse characteristics” a measurement time period for a sensor can be determined based on the pulse start control signal and the one or more laser pulse characteristics. For example, processor 202 can execute instructions 402 to determine measurement time period 216 based on laser pulse start signal 214 and laser pulse characteristics 212 where measurement time period 216 defines a period over which measurement by sensor 110 is to take place. As a specific example, processor 202 can execute instructions 402 to determine a period in which to modify sensor data 220 received at block 504.

Continuing to block 508 “generate, by the processor, modified sensor data based on the received sensor data and the measurement time period” modified sensor data can be generated by modifying the received sensor data based on the measurement time period. For example, processor 202 can execute instructions 402 to generate modified sensor data 404 from sensor data 220 based on measurement time period 216.

The method 500 can further include blocks (not shown) where the modified sensor data (or a graphical element comprising indications of the modified sensor data) is displayed on a display. For example, the method 500 can be implemented in real time, or rather, during a procedure, such that measurement of physical characteristics by sensor 110 of endoscope 106 can be made and them modified to synchronize the measurement with lasing by the lasing console 104 to reduce and/or mitigate interference in the with sensor measurements.

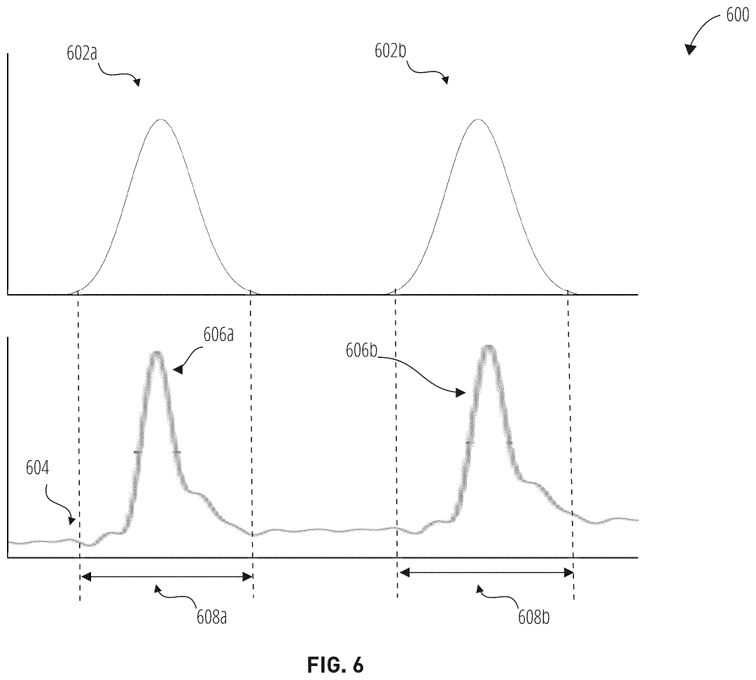

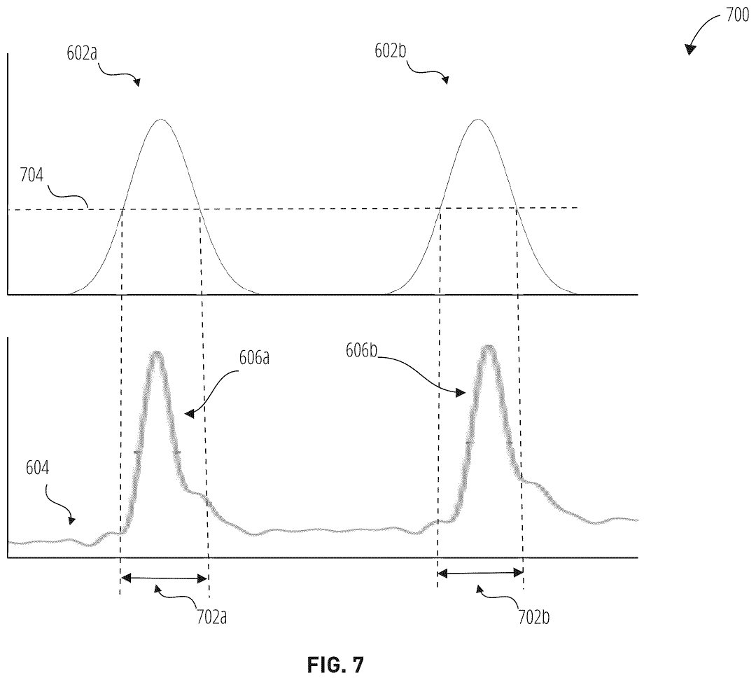

FIG. 6 and FIG. 7 illustrate an example of a measurement time period 216, which can be determined as outlined above. Both FIG. 6 and FIG. 7 illustrates plots 600 and 700, respectively, showing an intensity of a series of laser pulses 602a and 602b along with a corresponding sensor measurements 604. It is to be appreciated that laser pulses 602a and 602b are depicted as Gaussian for purposes of ease of illustration. However, is practice, the laser pulses 602a and 602b can have a variety of shapes or waveforms.

As can be seen, measurement spikes 606a and 606b correspond to the laser pulses 602a and 602b. These measurement spikes 606a and 606b can skew the average values for the measured physical condition (e.g., pressure, temperature, etc.) and/or can saturate the sensor electronics (e.g., sensor circuitry and mechanicals 222). As outlined above, the present disclosure provides to determine a measurement time period 216 in which either a sensor is to capture measurements of a physical condition of an environment 126 (e.g., FIG. 2 and FIG. 3) or in which already captured sensor measurements are to be modified. With some embodiments, as illustrated in FIG. 6 and plot 600, measurement time period 216 can be determined based on an initiation of laser pulses 602a and 602b. For example, this figure depicts measurement time periods 608a and 608b corresponding to periods in which laser pulses 602a and 602b are active.

Accordingly, in some embodiments, processor 202 can execute instructions 210 or instructions 402 to generate measurement time period 216 based on identifying an initiation and conclusion of laser pulses to be generated by lasing console 104.

In some embodiments, as illustrated in FIG. 7 and plot 700, measurement time period 216 can be determined based on laser pulses 602a and 602b exceeding a threshold level (e.g., a specified intensity level, or the like). For example, this figure depicts measurement time periods 702a and 702b corresponding to periods in which laser pulses 602a and 602b are above threshold 704. Accordingly, in some embodiments, processor 202 can execute instructions 210 or instructions 402 to generate measurement time period 216 based on identifying periods of time in which laser pulses to be generated by lasing console 104 will exceed a threshold level.

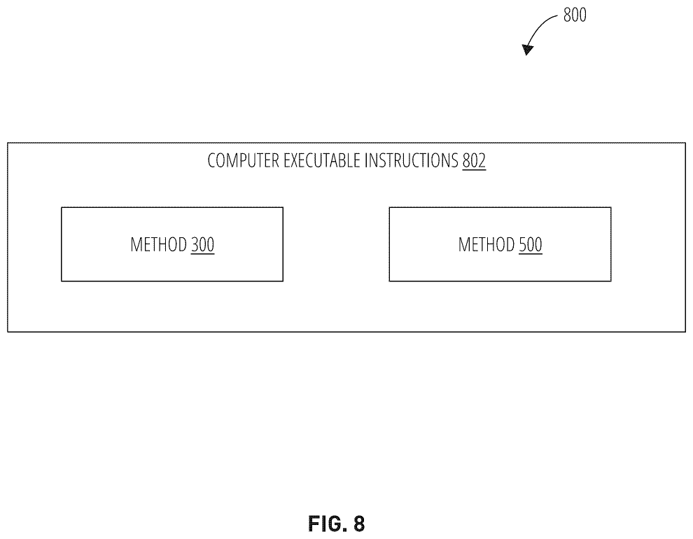

FIG. 8 illustrates computer-readable storage medium 800. Computer-readable storage medium 800 may comprise any non-transitory computer-readable storage medium or machine-readable storage medium, such as an optical, magnetic or semiconductor storage medium. In various embodiments, computer-readable storage medium 800 may comprise an article of manufacture. In some embodiments, computer-readable storage medium 800 may store computer executable instructions 802 with which circuitry (e.g., processor 202, or the like) can execute. For example, computer executable instructions 802 can include instructions to implement operations described with respect to method 300 and/or method 500. Examples of computer-readable storage medium 800 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 802 may include any suitable type of code, such as source code, compiled code, interpreted code, executable code, static code, dynamic code, object-oriented code, visual code, and the like.

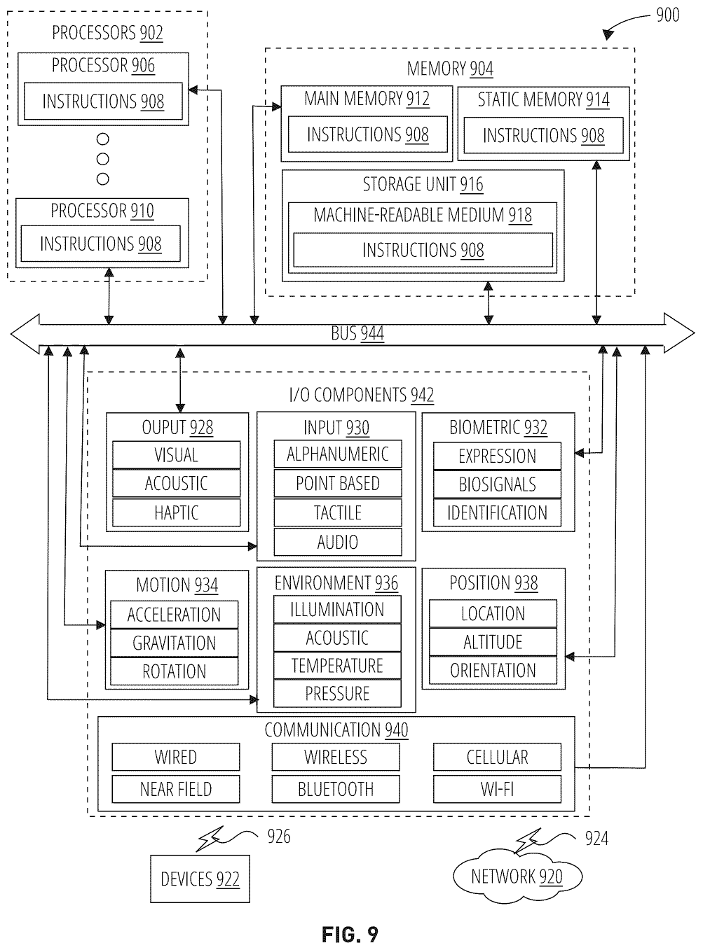

FIG. 9 illustrates a diagrammatic representation of a machine 900 in the form of a computer system within which a set of instructions may be executed for causing the machine to perform any one or more of the methodologies discussed herein. More specifically, FIG. 9 shows a diagrammatic representation of the machine 900 in the example form of a computer system, within which instructions 908 (e.g., software, a program, an application, an applet, an app, or other executable code) for causing the machine 900 to perform any one or more of the methodologies discussed herein may be executed. For example, the instructions 908 may cause the machine 900 to execute method 300 of FIG. 3, method 500 of FIG. 5, or the like. More generally, the instructions 908 may cause the machine 900 to synchronize measurement by an endoscopic sensor or measurements captured by an endoscopic sensor with lasing by a laser source.

The instructions 908 transform the general, non-programmed machine 900 into a particular machine 900 programmed to carry out the described and illustrated functions in a specific manner. In alternative embodiments, the machine 900 operates as a standalone device or may be coupled (e.g., networked) to other machines. In a networked deployment, the machine 900 may operate in the capacity of a server machine or a client machine in a server-client network environment, or as a peer machine in a peer-to-peer (or distributed) network environment. The machine 900 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 908, sequentially or otherwise, that specify actions to be taken by the machine 900. Further, while only a single machine 900 is illustrated, the term “machine” shall also be taken to include a collection of machines 200 that individually or jointly execute the instructions 908 to perform any one or more of the methodologies discussed herein.

The machine 900 may include processors 902, memory 904, and I/O components 942, which may be configured to communicate with each other such as via a bus 944. In an example embodiment, the processors 902 (e.g., a Central Processing Unit (CPU), a Reduced Instruction Sct Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 906 and a processor 910 that may execute the instructions 908. The term “processor” is intended to include multi-core processors that may comprise two or more independent processors (sometimes referred to as “cores”) that may execute instructions contemporaneously. Although FIG. 9 shows multiple processors 902, the machine 900 may include a single processor with a single core, a single processor with multiple cores (e.g., a multi-core processor), multiple processors with a single core, multiple processors with multiples cores, or any combination thereof.

The memory 904 may include a main memory 912, a static memory 914, and a storage unit 916, both accessible to the processors 902 such as via the bus 944. The main memory 904, the static memory 914, and storage unit 916 store the instructions 908 embodying any one or more of the methodologies or functions described herein. The instructions 908 may also reside, completely or partially, within the main memory 912, within the static memory 914, within machine-readable medium 918 within the storage unit 916, within at least one of the processors 902 (e.g., within the processor's cache memory), or any suitable combination thereof, during execution thereof by the machine 900.

The I/O components 942 may include a wide variety of components to receive input, provide output, produce output, transmit information, exchange information, capture measurements, and so on. The specific I/O components 942 that are included in a particular machine will depend on the type of machine. For example, portable machines such as mobile phones will likely include a touch input device or other such input mechanisms, while a headless server machine will likely not include such a touch input device. It will be appreciated that the I/O components 942 may include many other components that are not shown in FIG. 9. The I/O components 942 are grouped according to functionality merely for simplifying the following discussion and the grouping is in no way limiting. In various example embodiments, the I/O components 942 may include output components 928 and input components 930. The output components 928 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 930 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 942 may include biometric components 932, motion components 934, environmental components 936, or position components 938, among a wide array of other components. For example, the biometric components 932 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 934 may include acceleration sensor components (e.g., accelerometer), gravitation sensor components, rotation sensor components (e.g., gyroscope), and so forth. The environmental components 936 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 938 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 942 may include communication components 940 operable to couple the machine 900 to a network 920 or devices 922 via a coupling 924 and a coupling 926, respectively. For example, the communication components 940 may include a network interface component or another suitable device to interface with the network 920. In further examples, the communication components 940 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 922 may be another machine or any of a wide variety of peripheral devices (e.g., a peripheral device coupled via a USB).

Moreover, the communication components 940 may detect identifiers or include components operable to detect identifiers. For example, the communication components 940 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 940, such as location via Internet Protocol (IP) geolocation, location via Wi-Fi® signal triangulation, location via detecting an NFC beacon signal that may indicate a particular location, and so forth.

The various memories (i.e., memory 904, main memory 912, static memory 914, and/or memory of the processors 902) and/or storage unit 916 may store one or more sets of instructions and data structures (e.g., software) embodying or utilized by any one or more of the methodologies or functions described herein. These instructions (e.g., the instructions 908), when executed by processors 902, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 920 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 920 or a portion of the network 920 may include a wireless or cellular network, and the coupling 924 may be a Code Division Multiple Access (CDMA) connection, a Global System for Mobile communications (GSM) connection, or another type of cellular or wireless coupling. In this example, the coupling 924 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 908 may be transmitted or received over the network 920 using a transmission medium via a network interface device (e.g., a network interface component included in the communication components 940) and utilizing any one of several well-known transfer protocols (e.g., hypertext transfer protocol (HTTP)). Similarly, the instructions 908 may be transmitted or received using a transmission medium via the coupling 926 (e.g., a peer-to-peer coupling) to the devices 922. The terms “transmission medium” and “signal medium” mean the same thing and may be used interchangeably in this disclosure. The terms “transmission medium” and “signal medium” shall be taken to include any intangible medium that can store, encoding, or carrying the instructions 908 for execution by the machine 900, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

What is claimed is:

1. A method for an endoscope system, comprising:
receiving, at a processor of an endoscope console, an indication of a pulse start control signal and one or more laser pulse characteristics, the pulse start control signal to define an initiation of lasing by the lasing console and the one or more laser pulse characteristics to define the lasing;
determining, by the processor, a measurement time period for a sensor of the endoscope system based on the pulse start control signal and the one or more laser pulse characteristics; and
receiving by the processor or generating by the processor, sensor measurements based on the measurement time period.

2. The method of claim 1, wherein receiving by the processor sensor measurements based on the measurement time period comprising:
generating a control signal, the control signal to cause the sensor to measure a physical characteristic of an environment in which the endoscope is disposed based on the measurement time period; and
receiving, at the processor from the sensor, an information element comprising
indications of the physical characteristic measured based on the measurement time period.

3. The method of claim 2, wherein the control signal comprises an indication of the measurement time period and an indication to measure the physical characteristic outside the measurement time period.

4. The method of claim 2, wherein the control signal comprises an indication to power gate the sensor during the measurement time period.

5. The method of claim 1, wherein generating by the processor sensor measurements based on the measurement time period comprising:
receiving, at the processor from the sensor, an information element comprising indications of physical characteristic of an environment measured by the sensor; and
generating, by the processor, the sensor measurements based on the physical characteristics and the measurement time period.

6. The method of claim 5, wherein generating, by the processor, the sensor measurements based on the physical characteristics and the measurement time period comprising removing measurements captured during the measurement time period.

7. The method of claim 5, wherein generating, by the processor, the sensor measurements based on the physical characteristics and the measurement time period comprising normalizing measurements captured during the measurement time period based on measurements captured outside the measurement time period.

8. The method of claim 1, wherein the one or more laser pulse characteristics define a frequency of pulses to be generated by the lasing console.

9. The method of claim 1, wherein the one or more laser pulse characteristics define a pulse width of the pulses.

10. The method of claim 1, wherein determining the measurement time period comprising:
determining an initiation of one or more laser pulses based on the pulse start control signal;
determining a pulse width of the one or more laser pulses based on the one or more laser pulse characteristics;
determining a conclusion of the one or more laser pulses based on the pulse width; and
determining the measurement time period based on the initiation and conclusion of the one or more laser pulses.

11. The method of claim 1, wherein determining the measurement time period comprising:
determining a first instance where an intensity of one or more laser pulses exceeds a threshold value;
determining a second instance subsequent to the first instance where an intensity of the one or more laser pulses does not exceed the threshold value; and
determining the measurement time period based on the first instance and the second instance.

12. An endoscopic system comprising:
an endoscope comprising one or more working channels;
an endoscopic sensor; and
an endoscopic controller comprising a processor a memory comprising instructions, which when executed by the processor cause the processor to:
receive an indication of a pulse start control signal and one or more laser pulse characteristics, the pulse start control signal to define an initiation of lasing by the lasing console and the one or more laser pulse characteristics to define the lasing;
determine a measurement time period for a sensor of the endoscope system based on the pulse start control signal and the one or more laser pulse characteristics; and
receive by the processor or generating by the processor, sensor measurements based on the measurement time period.

13. The endoscope system of claim 6, the instructions, when executed by the processor, further cause the processor to:
generate a control signal, the control signal to cause the sensor to measure a physical characteristic of an environment in which the endoscope is disposed based on the measurement time period; and
receive an information element comprising indications of the physical characteristic measured based on the measurement time period.

14. The endoscope system of claim 13, wherein the control signal comprises an indication of the measurement time period and an indication to measure the physical characteristic outside the measurement time period.

15. The endoscope system of claim 13, wherein the control signal comprises an indication to power gate the sensor during the measurement time period.

16. The endoscope system of claim 12, the instructions, when executed by the processor, further cause the processor to:
receive, from the sensor, an information element comprising indications of physical characteristic of an environment measured by the sensor; and
generate the sensor measurements based on the physical characteristics and the measurement time period.

17. The endoscopic system of claim 6, the instructions, when executed by the processor, further cause the processor to remove measurements captured during the measurement time period.

18. A computer readable storage device comprising instructions that when executed by a processor of an endoscopic system cause the endoscopic system to:
receive an indication of a pulse start control signal and one or more laser pulse characteristics, the pulse start control signal to define an initiation of lasing by the lasing console and the one or more laser pulse characteristics to define the lasing;
determine a measurement time period for a sensor of the endoscope system based on the pulse start control signal and the one or more laser pulse characteristics; and
receive by the processor or generating by the processor, sensor measurements based on the measurement time period.

19. The computer readable storage device of claim 18, wherein the one or more laser pulse characteristics define a frequency of pulses to be generated by the lasing console.

20. The computer readable storage device of claim 18, wherein the one or more laser pulse characteristics define a pulse width of the pulses.
